(19) United States
(12) Patent Application Publication
(10) Pub. No.: US 2023/0312732 A1
(43) Pub. Date: Oct. 5, 2023

(54) EPHB2 ANTIBODY AND USE THEREOF IN COMBINATION THERAPY

(71) Applicant: 4C BIOMED SERVICES LTD., Netanya (IL)

(72) Inventors: Eyal GREENBERG, Even Yehuda (IL); Gilli GALORE-HASKEL, Yavne (IL); Efrat MERHAVI-SHOHAM, Rehovot (IL)

(73) Assignee: 4C BIOMED SERVICES LTD., Netanya (IL)

(21) Appl. No.: 18/011,291

(22) PCT Filed: Jun. 17, 2021
(86) PCT No.: PCT/IL2021/050740

§ 371 Date: Dec. 19, 2022

Related U.S. Application Data
(60) Provisional application No. 63/040,093, filed on Jun. 17, 2020.

(51) Int. Cl. C07K 16/28; C12N 15/113; A61K 39/395; A61P 35/00; A61K 35/17; C12N 5/0783

(52) U.S. Cl. CPC C07K 16/2866; C12N 15/1138; A61K 39/3955; C07K 16/2818; A61P 35/00; A61K 35/17; C12N 5/0636; C12N 2310/14; C07K 2317/76; C07K 2317/73; C12N 2501/599

(57) ABSTRACT

Methods of treating, preventing or ameliorating cancer by decreasing Ephrin type-B receptor 2 (EPHB2) function in a cancer cell and administering an immunotherapy are provided. Methods of enhancing an immunotherapy by decreasing EPHB2 function are also provided. Antibodies, pharmaceutical compositions and kits for performing the methods of the invention, and methods of producing those antibodies are also provided.

### CROSS REFERENCE TO RELATED APPLICATIONS

This application claims the benefit of priority of U.S. Provisional Patent Application No. 63/040,093, Jun. 17, 2020, the contents of which are all incorporated herein by reference in their entirety.

### FIELD OF INVENTION

The present invention is in the field of cancer therapy.

### BACKGROUND OF THE INVENTION

Immunotherapies designed to enhance an immune response are considered activating immunotherapies and are at the forefront of cancer treatment. Currently, immune checkpoint blockade therapy is successfully being used for treatment of advanced non-small cell lung cancers (NSCLC), metastatic melanoma, advanced renal cell carcinoma (RCC) and metastatic squamous cell carcinoma of the head and neck (SCCHN). A few such drugs, developed and manufactured by different companies, have shown great promise in different cancer types and have been FDA approved, including antibodies to the immune checkpoints programmed cell death protein 1 receptor (PD-1), programmed cell death protein 1 ligand (PD-L1) and cytotoxic T lymphocyte-associated protein 4 (CTLA-4). Patient response rate, however, is still not optimal, as only 10%-40% of treated patients usually benefit, and at the same time patients may suffer from Post Immunotherapy Treatments Side-Effects. In addition, treatment with these agents, may induce resistance through upregulation of additional immune checkpoints. Combination of anti-PD-1 and anti-CTLA-4 therapy in melanoma patients has demonstrated higher response rate (60%) as compared to single agent, however this combination therapy involves also severe treatment-related adverse effects.

Combination therapy is a major investigative avenue currently under examination for cancer therapy. Targeted therapies that aim at inhibiting a specific oncogene in combination with immune checkpoint blockade are of particular interest, though finding specific combinations with enhanced efficacy is challenging. Ephrin type-B receptor 2 (EPHB2), also known as HEK5, is a major receptor of Ephrin B type ligands and also Ephrin A type ligands. It has been reported that the Eph receptor-Ephrin system is overexpressed and deregulated in a variety of tumors, involved in tumorigenesis, tumor angiogenesis and metastasis development. For example, siRNA knockdown of EPHB2 in cervical carcinoma cells in culture inhibited proliferation and metastasis. Moreover, Eph receptors and Ephrins are expressed on a variety of immune cells and have been implicated in various aspects of immune surveillance such as immune cell activation, migration, adhesion and proliferation. An effective therapy that can target EPHB2 in cancer patients, as well as an effective combination therapy making use of EPHB2 targeting is greatly needed.

### SUMMARY OF THE INVENTION

The present invention provides methods of treating, preventing or ameliorating cancer by decreasing Ephrin type-B receptor 2 (EPHB2) function in a cancer cell and administering an immunotherapy. Methods of enhancing an immunotherapy by decreasing EPHB2 function, as well as antibodies, pharmaceutical compositions and kits for performing the methods of the invention are also provided.

According to a first aspect, there is provided a method of treating, preventing or ameliorating cancer in a subject in need thereof, the method comprising:
a. decreasing Ephrin type-B receptor 2 (EPHB2) function in a cell of the cancer; and
b. administering to the subject an immunotherapy,
thereby treating, preventing or ameliorating cancer in a subject.

According to another aspect, there is provided a method of enhancing an immunotherapy in a subject in need thereof, the method comprising decreasing Ephrin type-B receptor 2 (EPHB2) function in the subject, thereby enhancing an immunotherapy in a subject.

According to some embodiments, the subject suffers from cancer.

According to some embodiments, the cancer is a solid tumor.

According to some embodiments, the cancer is treatable by the immunotherapy.

According to some embodiments, the cancer expresses EPHB2.

According to some embodiments, the cancer is selected from skin cancer, kidney cancer and colon cancer.

According to some embodiments, the cancer is selected from melanoma and renal cell carcinoma (RCC).

According to some embodiments, the subject is naïve to immunotherapy, has been previously treated by the immunotherapy, or has been treated by an immunotherapy other than the immunotherapy.

According to some embodiments, the decreasing EPHB2 function comprises decreasing EPHB2 expression in the cell of the cancer.

According to some embodiments, the decreasing expression comprises administering a pharmaceutical composition comprising a regulatory nucleic acid molecule that bind to and inhibits a EPHB2 mRNA or the EPHB2 genomic locus.

According to some embodiments, the decreasing function comprises administering a pharmaceutical composition comprising an agent that binds to EPHB2 on a surface of a cancer cell.

According to some embodiments, binding of the agent to EPHB2 induces reduced expression of EPHB2 on the cell surface, induces degradation of the EPHB2, blocks signaling through the EPHB2, blocks binding of the EPHB2 to a ligand, and/or blocks signaling through an EPHB2 ligand.

According to some embodiments, the ligand is type B ephrin or a type A ephrin.

According to some embodiments, the ligand is selected from Ephrin B1 (EFNB1), Ephrin B3 (EFNB3) and Ephrin A5 (EFNA5).

According to some embodiments, the agent is an EPHB2 blocking antibody.

According to some embodiments, the agent is not coupled to a cytoxic moiety.

According to some embodiments, the immunotherapy comprises immune checkpoint blockade.

According to some embodiments, the immune checkpoint blockade is administered to the subject or wherein ex vivo immune cells are treated with the immune checkpoint blockade and the treated immune cells are administered to the subject.

According to some embodiments, the immune cells are selected from peripheral blood mononuclear cells (PBMCs), T cells, tumor infiltrating lymphocytes (TILs), natural killer cells (NK cells) and macrophages.

According to some embodiments, the administering treated immune cells is adoptive T cell therapy.

According to some embodiments, the immune checkpoint blockade comprises administering a pharmaceutical composition comprising an antibody that binds to and inhibits PD-1, PD-L1, PD-L2, CD80, CD86, VISTA, CD275, CD276, VTCN1, HHLA2, CD96, CD155, TIGIT, CD112R, CD112, CD200, CD200R, CTLA4, LAG3, FGL1, TIM3, CEACAM-1, Gal-9, HMGB1, Butyrophilin family members, HVEM, or BTLA.

According to some embodiments, the pharmaceutical composition comprises an antibody that binds to and inhibits PD-1.

According to some embodiments, the immunotherapy is anti-PD-1 antibody therapy.

According to some embodiments, the decreasing EPHB2 function improves the efficacy of the immunotherapy.

According to some embodiments, the treating, preventing or ameliorating cancer or the enhancing immunotherapy comprises increasing immune activation of an immune cell in said subject.

According to some embodiments, the immune cell is a TIL.

According to another aspect, there is provided a kit comprising
a. an antibody that binds to EPHB2 on a cell and inhibits a function of said EPHB2 or a pharmaceutical composition comprising said antibody; and
b. a pharmaceutical composition comprising an immunotherapy.

According to some embodiments, the kit further comprises a label stating the antibody or pharmaceutical composition comprising the antibody is for use with the immunotherapy.

According to some embodiments, the immunotherapy comprises immune checkpoint blockade.

According to another aspect, there is provided a method for producing an agent that enhances an immunotherapy, the method comprising:
obtaining an agent that binds to a EPHB2 extracellular domain or a fragment thereof, assaying the efficacy of an immunotherapy on cancer cells treated with the agent, and selecting at least one agent that enhances the efficacy of the immunotherapy; or
culturing a host cell comprising one or more vectors comprising a nucleic acid sequence encoding an agent, wherein the nucleic acid sequence is that of an agent that was selected by:
   i. obtaining an agent that binds to a EPHB2 extracellular domain or a fragment thereof;
   ii. assaying the efficacy of an immunotherapy on cancer cells treated with the agent; and
   iii. selecting at least one agent that enhances the efficacy of the immunotherapy;
thereby producing an agent that enhances an immunotherapy.

According to some embodiments, the method further comprised assaying EPHB2 function in the presence of the obtained agent and selecting an agent that inhibits EPHB2 function.

According to some embodiments, the EPHB2 function is EPHB2 downstream signaling, EPHB2 ligand binding or downstream signaling of an EPHB2 ligand.

According to some embodiments, the ligand is selected from a B class ephrin and an A class ephrin.

According to some embodiments, the ligand is selected from Ephrin B1 (EFNB1), Ephrin B3 (EFNB3) and Ephrin A5 (EFNA5).

According to some embodiments, the immunotherapy comprises immune checkpoint blockade.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B: (1A-1B) Bar graphs of (1A) % specific cell killing of melanoma cells and RCC cells by autologous TILs pre-treated with anti-PD-1 antibody, and (1B) relative cell number of melanoma cells and RCC cells in the absence of TILs but in the presence of anti-PD-1, after transfection of the cancerous cells with a control (black bars) non-targeting siRNA or an anti-EPHB2 (grey bars) siRNA.

FIGS. 2A-2D: (2A-2C) Line graphs of (2A, 2C) Caspase 3/7 positive events in melanoma cells pre-treated with an anti-EPHB2 antibody (grey lines) or an isotype control (black lines) and cocultured with (2A) anti-PD-1 antibody pretreated TILs or (2C) TILs (in the absence of anti-PD-1) and (2B) 41BB expression of the TILs from 2A. (2D) Bar graph of % Caspase 3/7 positive events in melanoma cells 25 hours after treatment with anti-EPHB2 antibody or an isotype control in the presence of anti-PD-1 antibody but in the absence of TILs.

FIG. 3: A line graph of Caspase 3/7 positive events in colon carcinoma cells cultured with an anti-EPHB2 antibody (grey lines) or an isotype control (black lines) and anti-PD-1 antibody and then cocultured with autologous PBMCs.

FIG. 4: Bar graph of % increase in expression of EPHB2 ligands in TILs after exposure to anti-PD-1 antibody.

### DETAILED DESCRIPTION OF THE INVENTION

The present invention, in some embodiments, provides methods of treating, preventing and/or ameliorating cancer, as well as methods of improving/enhancing immunotherapy. The present invention further concerns antibodies, pharmaceutical compositions and kits for performing the methods of the invention. A method of producing the antibodies of the invention is also provided.

By a first aspect, there is provided a method of treating, preventing or ameliorating cancer in a subject in need thereof, the method comprising decreasing Ephrin type-B receptor 2 (EPHB2) function in a cell of said cancer, thereby treating, preventing or ameliorating cancer in a subject.

By another aspect, there is provided a method of enhancing an immunotherapy in a subject in need thereof, the method comprising decreasing Ephrin type-B receptor 2 (EPHB2) function in the subject, thereby enhancing an immunotherapy in a subject.

By another aspect, there is provided a method of increasing immune activation of an immune cell, the method comprising decreasing Ephrin type-B receptor 2 (EPHB2) function in a cancer cell in contact with said immune cell and contacting said immune cell with an immunotherapy.

In some embodiments, the method is a method of treating cancer. In some embodiments, the method is a method of preventing cancer. In some embodiments, the method is a method of ameliorating cancer. In some embodiments, the method is a combination therapy. In some embodiments, the method is an enhanced immunotherapy. In some embodiments, enhanced is improved. In some embodiments, the method is for increasing immune activation. In some embodiments, treating, preventing, ameliorating or enhancing comprises increasing immune activation in an immune cell. In some embodiments, immune activation is activation of an immune cell. Increased activation can be determined by any method known in the art, or with any known activation marker. An example of such a marker that can be measured is 41BB expression. In some embodiments, increased immune activation comprises increased 41BB expression. In some embodiments, the immune cell is a PBMC. In some embodiments, the immune cell is a T cell. In some embodiments, the immune cell is a TIL.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, EPHB2 is mammalian EPHB2. In some embodiments, the mammal is a rodent. In some embodiments, the rodent is a mouse or a rat. In some embodiments, the mammal is human. In some embodiments, the human EPHB2 gene is provided in Entrez Gene 2048. In some embodiments, the human EPHB2 protein is encoded by an mRNA sequence selected from NM_001309192, NM_001309193, NM_004442 and NM_017449. In some embodiments, the human EPHB2 protein is encoded by the mRNA sequence NM_017449 In some embodiments, the human EPHB2, protein is provided in UniProt number P29323. In some embodiments, the human EPHB2 protein comprises or consist of the amino acid sequence selected from NP 001296121, NP 001296122, NP_004433 and NP_059145. In some embodiments, the human EPHB2 protein comprises or consist of the amino acid sequence of NP_059145.

In some embodiments, the subject is a mammal. In some embodiments, the subject is a human. In some embodiments, the subject suffers from cancer. In some embodiments, the subject is naïve to cancer treatment. In some embodiments, the subject is naïve to immunotherapy. In some embodiments, the subject has received treatment. In some embodiments, the subject is refractive to treatment. In some embodiments, the treatment is an immunotherapy. In some embodiments, the treatment is the immunotherapy. In some embodiments, the treatment is an immunotherapy other than the immunotherapy. In some embodiments, the treatment is selected from radiotherapy, chemotherapy, targeted therapy and immunotherapy. In some embodiments, the subject was previously administered a therapy and the therapy was found ineffective. In some embodiments, the subject was previously administered a therapy, the therapy was initially effective but has become ineffective or less effective.

In some embodiments, the cancer is a solid tumor. In some embodiments, the cancer is a hematopoietic cancer. In some embodiments, the cancer is treatable by immunotherapy. In some embodiments, the cancer expresses an immune checkpoint inhibitory molecule. In some embodiments, the cancer overexpresses an immune checkpoint inhibitory molecule. In some embodiments, the immune checkpoint inhibitory molecule is a checkpoint inhibitory ligand. In some embodiments, the immune checkpoint inhibitory molecule is selected from PD-L1, PD-L2, CD80, CD86, CD275, CD276, VTCN1, VISTA, HHLA2, HVEM, FGL1, CD155, CD112, CD200, HMGB1, Butyrophilin family members and Gal-9. In some embodiments, the cancer is a PD-L1 positive cancer. In some embodiments, the cancer is a PD-L2 positive cancer. In some embodiments, the cancer is an HVEM positive cancer.

In some embodiments, the cancer is skin cancer. In some embodiments, the cancer is melanoma. In some embodiments, the cancer is kidney cancer. In some embodiments, the cancer is renal cell carcinoma (RCC). In some embodiments, the cancer is colon cancer. In some embodiments, the cancer is colon carcinoma. In some embodiments, cancer is selected from breast cancer, cervical cancer, endocervical cancer, colon cancer, rectal cancer, bladder cancer, lymphoma, esophageal cancer, brain cancer, head and neck cancer, renal cancer, bile duct cancer, meningeal cancer, glioma, glioblastoma, sarcoma, Langerhans cell cancer, leukemia, lung cancer, mesothelioma, ovarian cancer, pancreatic cancer, adrenal cancer, neuroendocrine cancer, prostate cancer, skin cancer, gastric cancer, tenosynovial cancer, tongue cancer, thyroid cancer, uterine cancer, and testicular cancer. In some embodiments, cancer is selected from breast cancer, endocervical cancer, colon cancer, rectal cancer, bladder cancer, lymphoma, esophageal cancer, brain cancer, head and neck cancer, renal cancer, bile duct cancer, meningeal cancer, glioma, glioblastoma, sarcoma, Langerhans cell cancer, leukemia, lung cancer, mesothelioma, ovarian cancer, pancreatic cancer, adrenal cancer, neuroendocrine cancer, prostate cancer, skin cancer, gastric cancer, tenosynovial cancer, tongue cancer, thyroid cancer, uterine cancer, and testicular cancer. In some embodiments, cancer is selected from breast cancer, colon cancer, rectal cancer, bladder cancer, lymphoma, esophageal cancer, brain cancer, head and neck cancer, renal cancer, bile duct cancer, meningeal cancer, glioma, glioblastoma, sarcoma, Langerhans cell cancer, leukemia, lung cancer, mesothelioma, ovarian cancer, pancreatic cancer, adrenal cancer, neuroendocrine cancer, prostate cancer, skin cancer, gastric cancer, tenosynovial cancer, tongue cancer, thyroid cancer, uterine cancer, and testicular cancer. In some embodiments, the renal cancer is renal cell carcinoma. In some embodiments, the skin cancer is melanoma. In some embodiments, the colon cancer is colon carcinoma. In some embodiments, the rectal cancer is rectal adenocarcinoma. In some embodiments, the bile duct cancer is cholangiocarcinoma. In some embodiments, the adrenal cancer is adrenocortical carcinoma. In some embodiments, the cancer is selected from skin cancer, kidney cancer and colon cancer. In some embodiments, the cancer is selected from melanoma, RCC and colon carcinoma. In some embodiments, the cancer is not cervical cancer. In some embodiments, the cancer is not cervical carcinoma.

In some embodiments, the cancer is an EPHB2 positive cancer. In some embodiments, the cancer expresses EPHB2. In some embodiments, the cancer overexpresses EPHB2. In some embodiments, the cancer secretes soluble EPHB2.

In some embodiments, decreasing EPHB2 function comprises decreasing EPHB2 expression. In some embodiments, decreasing EPHB2 function comprises decreasing EPHB2 levels. In some embodiments, EPHB2 levels are protein levels. In some embodiments, EPHB2 levels are mRNA levels. In some embodiments, decreasing EPHB2 function comprises decreasing EPHB2 transcription. In some embodiments, decreasing EPHB2 function comprises decreasing EPHB2 translation. In some embodiments, decreasing expression comprises administering a nucleic acid molecule that inhibits EPHB2. In some embodiments, the nucleic acid molecule is a regulatory nucleic acid molecule. In some embodiments, the nucleic acid molecule is a guide RNA. In some embodiments, decreasing EPHB2 function comprises administering a genome editing protein or complex. In some embodiments, decreasing expression comprises administering a regulatory nucleic acid molecule that inhibits EPHB2.

In some embodiments, the nucleic acid molecule is an RNA. In some embodiments, decreasing expression comprises administering a regulatory RNA that inhibits EPHB2. In some embodiments, the nucleic acid molecule is an antisense oligonucleotide. In some embodiments, the nucleic acid molecule is a non-naturally occurring nucleic acid molecule. In some embodiments, the nucleic acid molecule is an artificial nucleic acid molecule. In some embodiments, the nucleic acid molecule comprises a chemical modification. In some embodiments, the chemical modification is a chemically modified backbone. In some embodiments, the chemically modified backbone comprises at least one of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense and a combination thereof. Each possibility represents a separate embodiment of the invention.

In some embodiments, the regulatory molecule binds to a EPHB2 mRNA. In some embodiments, the molecule binds to the EPHB2 genomic locus. In some embodiments, the molecule targets a genome editing protein or complex to the EPHB2 genomic locus. In some embodiments, the regulatory molecule binds to a EPHB2 regulatory domain. In some embodiments, the regulatory domain is the promoter. In some embodiments, the regulatory molecule is in a pharmaceutical composition. In some embodiments, the regulatory molecule is in an expression vector. In some embodiments, the expression vector is configured for expression in the cancer cell. In some embodiments, the expression vector is configured for expressing the regulatory molecule. In some embodiments, decreasing expression comprises administering a pharmaceutical composition. In some embodiments, the pharmaceutical composition comprises the regulatory molecule. In some embodiments, the pharmaceutical composition comprises an expression vector comprising the regulatory molecule. In some embodiments, the regulatory RNA is a short inhibitory RNA (siRNA). In some embodiments, the regulatory RNA is RNAi. In some embodiments, the regulatory RNA is a short hairpin RNA (shRNA). In some embodiments, the regulatory RNA is microRNA (miR). MiRs that target EPHB2 are known in the art and include, for example, miR-204; any such miR may be used.

In some embodiments, genome editing protein edits the EPHB2 genomic locus such that EPHB2 is no longer expressed. In some embodiments, genome editing protein ablates a portion of the EPHB2 genomic locus. In some embodiments, genome editing protein introduces a mutation into the EPHB2 genomic locus. In some embodiments, the genome editing protein introduces a deletion into the EPHB2 genomic locus. Genome editing proteins/complexes are well known in the art and any such protein/complex may be employed for editing the EPHB2 genomic locus. In some embodiments, a genome editing protein is selected from the group consisting of a clustered Regularly Interspaced Short Palindromic Repeats (CRISPR)-associated nuclease, a Zinc-finger nuclease (ZFNs), a meganuclease and a transcription activator-like effector nuclease (TALEN). In some embodiments, the decreasing comprises expressing the genome editing protein in the subject. In some embodiments, the decreasing comprises expressing the genome editing protein in the cancer cell.

Expressing of a molecule within a cell is well known to one skilled in the art. It can be carried out by, among many methods, transfection, viral infection, or direct administration. In some embodiments, the gene is in an expression vector such as plasmid or viral vector. In some embodiments, vector is withing an appropriate composition for allowing entrance of the vector into cells. For example, placing the vector within a liposome or micelle will allow for uptake of the vector into cells.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter. The promoter may be active in cancer cells.

In some embodiments, the regulatory RNA is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2 (±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, intratumor or intraperitoneal. In some embodiments, a therapeutically effective dose is administered. In some embodiments, a therapeutically effective dose of immunotherapy is administered. In some embodiments, a therapeutically effective dose of a pharmaceutical composition is administered. In some embodiments, a therapeutically effective dose of a pharmaceutical composition of the invention is administered. In some embodiments, a therapeutically effective dose of an agent of the invention is administered.

The dosage administered will be dependent upon the age, health, and weight of the recipient, kind of concurrent treatment, if any, frequency of treatment, and the nature of the effect desired.

In some embodiments, decreasing function comprises administering an agent that binds to EPHB2 protein. In some embodiments, the agent is in a pharmaceutical composition. In some embodiments, the agent is an agent of the invention. In some embodiments, the agent is an antibody of the invention. In some embodiments, the agent is an antibody. In some embodiments, the pharmaceutical composition is a pharmaceutical composition of the invention. In some embodiments, the agent is a small molecule. In some embodiments, the agent is a small molecule inhibitor. In some embodiments, the pharmaceutical composition comprises the antibody. In some embodiments, the pharmaceutical composition comprises the agent. In some embodiments, the agent binds to EPHB2 on a cell surface. In some embodiments, the agent binds to EPHB2 on the surface of a cancer cell. In some embodiments, the agent binds to soluble EPHB2. In some embodiments, binding of the agent induces reduced expression of EPHB2. In some embodiments, reduced expression is reduced surface expression. In some embodiments, binding of the agent induces internalization of EPHB2. In some embodiments, binding of the agent induces degradation of EPHB2. In some embodiments, binding of the agent reduces EPHB2 function. In some embodiments, binding of the agent inhibits EPHB2 function. In some embodiments, binding of the agent blocks a ligand binding domain. In some embodiments, binding of the agent blocks or reduces binding of EPHB2 to a ligand. In some embodiments, the agent binds a ligand binding domain of EPHB2. In some embodiments, the agent binds outside a ligand binding domain and occludes the ligand binding domain. In some embodiments, the agent binds outside a ligand binding domain and induces a conformational change that inhibits binding to the ligand binding domain. In some embodiments, binding of the agent reduces signaling through EPHB2. In some embodiments, binding of the agent inhibits signaling through EPHB2. In some embodiments, binding of the agent modulates signaling through EPHB2. In some embodiments, binding of the agent reduces signaling through an EPHB2 ligand. In some embodiments, binding of the agent inhibits signaling through an EPHB2 ligand. In some embodiments, binding of the agent modulates signaling through an EPHB2 ligand.

In some embodiments, the ligand is a B class ephrin. In some embodiments, the ligand is an A class ephrin. In some embodiments, the ligand is selected from a B class ephrin and an A class ephrin. In some embodiments, the B class ephrin is selected from EFNB1, EFNB2, and EFNB3. In some embodiments, the B class ephrin is selected from EFNB1 and EFNB3. In some embodiments, the B class ephrin is EFNB1. In some embodiments, the B class ephrin is EFNB2. In some embodiments, the B class ephrin is EFNB3. In some embodiments, the B class ephrin is a human ephrin. In some embodiments, the A class ephrin is selected from EFNA1, EFNA2, EFNA3, EFNA4, and EFNA5. In some embodiments, the A class ephrin is EFNA1. In some embodiments, the A class ephrin is EFNA2. In some embodiments, the A class ephrin is EFNA3. In some embodiments, the A class ephrin is EFNA4. In some embodiments, the A class ephrin is EFNA5. In some embodiments, the A class ephrin is a human ephrin. In some embodiments, the ligand is selected from Ephrin B1 (EFNB1), Ephrin B3 (EFNB3) and Ephrin A5 (EFNA5). In some embodiments, the ligand is selected from Ephrin B1 (EFNB1), Ephrin B2 (EFNB2), Ephrin B3 (EFNB3) and Ephrin A5 (EFNA5).

In some embodiments, the agent does not comprise a cytotoxic moiety. In some embodiments, the agent does not comprise a toxic agent. In some embodiments, a toxic agent or cytotoxic moiety is a molecule that kills the cell when internalized. In some embodiments, the cytotoxic moiety or toxic agent is not an antibody constant region. In some embodiments, the agent is not configured to deliver a toxic agent to the inside of a cell. In some embodiments, the agent is not internalized upon binding to EPHB2. In some embodiments, the agent does not induce cancer cell death when administered as a monotherapy. In some embodiments, the agent does not induce significant cancer cell death when administered as a monotherapy.

In some embodiments, the method further comprises administering an immunotherapy to the subject. In some embodiments, the immunotherapy is a therapy that does not decrease EPHB2 function. In some embodiments, the immunotherapy is a different immunotherapy. In some embodiments, the immunotherapy is a targeted immunotherapy. As used herein, the term “targeted immunotherapy” refers to an immunotherapy that strives to induce a specific immune response to a tumor antigen. A target immunotherapy therefore induces an immune response targeted to the cancer. Examples of targeted immunotherapy include, but are not limited to, cytotoxic antibodies and chimeric antigen receptor (CAR) expressing cells with a CAR that targets a tumor antigen. Examples of tumor antigens that are currently targeted by immunotherapeutics include, for example, CD52 (alemtuzumab), EGFR (cetuximab/panitumumab), CD20 (rituximab), MUC1, and Her2 (trastuzumab).

In some embodiments, the immunotherapy is an immune cell transplant. In some embodiments, the immunotherapy is adoptive T cell therapy. In some embodiments, the T cell is a tumor infiltrating lymphocyte (TIL). In some embodiments, the T cell is a CD8 positive T cell. In some embodiments, the T cell is a cytotoxic T cell. In some embodiments, the immune cell is an autologous immune cell. In some embodiments, the immune cell is non-autologous immune cell. In some embodiments, the immune cell is a syngeneic immune cell. In some embodiments, the immune cell is an allogenic immune cell. In some embodiments, the immune cell is peripheral blood mononuclear cells (PBMCs). In some embodiments, the immune cell is selected from a T cell, a B cell, a macrophage, and a natural killer cell (NK cell). In some embodiments, the immune cell is selected from a T cell, a macrophage, and a natural killer cell (NK cell). In some embodiments, the immune cell is a T cell. In some embodiments, the immune cell is a CAR cell. In some embodiments, the CAR cell is selected from a CAR-T cell and a CAR-NK cell.

In some embodiments, the immunotherapy is immune checkpoint blockade. Immune checkpoint blockade is well known in the art and is a method for activating immune surveillance that may have been avoided by a cancer expressing an immune inhibitory ligand. The blockade may activate immune cells, increase immune cell proliferation, make the tumor cells detectable by the immune system or a combination of the above. In some embodiments, immune checkpoint blockade is immune checkpoint inhibition. In some embodiments, immune checkpoint blockade comprises administering an antibody that binds to an immune checkpoint protein. In some embodiments, the antibody is in a pharmaceutical composition. In some embodiments, the immune checkpoint blockade comprises administering the pharmaceutical composition. In some embodiments, the checkpoint protein is a receptor on the immune cell. In some embodiments, the checkpoint protein is a ligand expressed by the cancer cell. In some embodiments, the antibody inhibits the immune checkpoint protein. In some embodiments, the immune checkpoint protein is selected from PD-1, PD-L1, PD-L2, CD80, CD86, VISTA, CD275, CD276, VTCN1, HHLA2, CD96, CD155, TIGIT, CD112R, CD112, CD200, CD200R, CTLA4, LAG3, FGL1, TIM3, CEACAM-1, Gal-9, HMGB1, Butyrophilin family members, HVEM, and BTLA. In some embodiments, the immune checkpoint protein is PD-1. In some embodiments, the immune checkpoint protein is PD-L1. In some embodiments, the immune checkpoint protein is HVEM.

In some embodiments, the immunotherapy is anti-PD-1 therapy. In some embodiments, the immunotherapy is anti-PD-1 antibody therapy. In some embodiments, the immunotherapy is anti-PD-1 immune checkpoint blockade. In some embodiments, the immunotherapy is PD-1 blockade. In some embodiments, the immunotherapy is anti-PD-1/PD-L1/PD-L2 therapy. In some embodiments, the immunotherapy is anti-PD-1/PD-L1/PD-L2 antibody therapy. In some embodiments, the immunotherapy is anti-PD-1/PD-L1/PD-L2 immune checkpoint blockade. In some embodiments, the immunotherapy is PD-1/PD-L1/PD-L2 blockade. In some embodiments, the immunotherapy is anti-PD-1/PD-L1 therapy. In some embodiments, the immunotherapy is anti-PD-1/PD-L1 antibody therapy. In some embodiments, the immunotherapy is anti-PD-1/PD-L1 immune checkpoint blockade. In some embodiments, the immunotherapy is PD-1/PD-L1 blockade.

In some embodiments, the immunotherapy comprises one of the therapies provided hereinabove. In some embodiments, the immunotherapy is a combination immunotherapy comprising at least two immunotherapies. In some embodiments, the combination immunotherapy is immune checkpoint blockade and immune cell transfer. In some embodiments, the immunotherapy is administered to the subject. In some embodiments, the immune checkpoint inhibitor is administered to the subject. In some embodiments, the immunotherapy is applied to ex vivo immune cells. In some embodiments, ex vivo immune cells are treated with the immunotherapy. In some embodiments, the ex vivo immune cells are treated with an immune checkpoint inhibitor that binds to an immune receptor on the immune cells. It will be understood by a skilled artisan that when the checkpoint inhibitor is administered to the ex vivo cells to be transferred, it cannot be a molecule that binds the ligand on the cancer cells, but rather must target the receptor on the immune cells. In some embodiments, the treated immune cells are administered to the subject. In some embodiments, the immune cells are in a pharmaceutical composition. In some embodiments, the pharmaceutical composition is administered to the subject. In some embodiments, the administering the treated immune cells is adoptive T cell therapy.

In some embodiments, decreasing EPHB2 function improves the efficacy of the immunotherapy. In some embodiments, improving efficacy is enhancing the immunotherapy. In some embodiments, enhancement or improvement comprises at least a 10, 20, 25, 30, 40, 50, 60, 70, 75, 80, 90, 100, 150, 200, 250, 300, 350, 400, 450, or 500% increase in efficacy. Each possibility represents a separate embodiment of the invention. In some embodiments, efficacy is measured by specific cell killing. In some embodiments, cell killing is cancer cell killing. In some embodiments, the increase is in the number of killed tumor cells. In some embodiments, efficacy is measured in tumor size. In some embodiments, efficacy is measured in time until relapse. In some embodiments, improved efficacy or enhancement is converting a non-effective immunotherapy to an effective immunotherapy. In some embodiments, improved efficacy or enhancement is converting an immunotherapy to which the patient is refractive to a once again effective therapy.

According to another aspect, there is provided an agent that binds to EPHB2 and inhibits a function of the EPHB2.

In some embodiments, the agent binds to EPHB2 on a cell surface. In some embodiments, the agent binds to EPHB2 on a surface of a cancer cell. In some embodiments, the agent binds to soluble EPHB2. In some embodiments, the agent binds to a ligand binding domain of EPHB2. In some embodiments, the agent blocks binding of a ligand to EPHB2. In some embodiments, the ligand is B type ephrin. In some embodiments, the ligand is an A type ligand. In some embodiments, the agent blocks binding of a first ligand and not binding of a second ligand. In some embodiments, the first ligand is a B type ephrin. In some embodiments, the first ligand is an A type ligand. In some embodiments, the second ligand is a B type ligand. In some embodiments, the second ligand is an A type ligand. In some embodiments, the agent does not block binding of a non-B type ephrin ligand to EPHB2. In some embodiments, the agent occludes a ligand binding domain. In some embodiments, the agent binds to a non-ligand binding domain and inhibits binding of a ligand to the ligand binding domain. In some embodiments, the agent binds to an extracellular domain of EPHB2. In some embodiments, the agent binds to a fragment of the extracellular domain of EPHB2. In some embodiments, the agent does not activate EPHB2. In some embodiments, the agent inhibits downstream signaling through EPHB2. In some embodiments, the agent inhibits downstream signaling through an EPHB2 ligand. In some embodiments, the agent blocks downstream signaling through EPHB2. In some embodiments, the agent blocks downstream signaling through an EPHB2 ligand.

In some embodiments, the agent is an antibody. In some embodiments, the agent is a small molecule. In some embodiments, the agent is a protein. In some embodiments, the antibody is a monoclonal antibody. In some embodiments, the antibody is a humanized antibody. In some embodiments, the antibody is a human antibody. In some embodiments, the antibody is a single-chain antibody. In some embodiments, the antibody is a single-domain antibody. In some embodiments, the antibody is a cytotoxic antibody. In some embodiments, the antibody is a non-cytotoxic antibody. In some embodiments, the antibody comprises an IgG backbone. In some embodiments, the antibody comprises an IgG constant region. In some embodiments, the IgG is IgG1. In some embodiments, the IgG is IgG3. In some embodiments, the IgG is IgG1 or IgG3. In some embodiments, the IgG is IgG2 or IgG4. In some embodiments, the IgG is an IgG with enhanced cytotoxicity. In some embodiments, the IgG is an IgG with reduced cytotoxicity.

In some embodiments, the agent enhances the efficacy of an immunotherapy. In some embodiments, the agent has a synergistic effect with an immunotherapy. In some embodiments, the immunotherapy is immune checkpoint blockade. In some embodiments, the agent is not an immunotherapy. In some embodiments, the agent is an immunotherapy. In some embodiments, the agent enhances an immune response against the cancer. In some embodiments, the agent enhances a general immune response. In some embodiments, the agent enhances immune targeting to the cancer cells and also inhibits EPHB2 diminishing cancer cell health. In some embodiments, diminishing cancer cell health is decreasing proliferation. In some embodiments, diminishing cancer cell health is decreasing metastasis. In some embodiments, diminishing cancer cell health is decreasing cell viability. In some embodiments, diminishing cancer cell health is increasing cancer cell apoptosis.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass. In some embodiments, the antibody comprises IgG2 or IgG4. In some embodiments, the antibody comprises IgG2. In some embodiments, the antibody comprises IgG4. In some embodiments, the antibody comprises IgG1. In some embodiments, the antibody comprises IgG3. In some embodiments, the antibody comprises a modified IgG1 or IgG3 with reduced toxicity.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e. CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.).

Immunoglobulin variable domains can also be analyzed using the IMGT information system (www://imgt.cines.fr/) (IMGT®/V-Quest) to identify variable region segments, including CDRs. See, e.g., Brochet, X. et al, Nucl. Acids Res. J6:W503-508 (2008).

As used herein, the term “humanized antibody” refers to an antibody from a non-human species whose protein sequences have been modified to increase similarity to human antibodies. A humanized antibody may be produced by production of recombinant DNA coding for the CDRs of the non-human antibody surrounded by sequences that resemble a human antibody. In some embodiments, the humanized antibody is a chimeric antibody. In some embodiments, humanizing comprises insertion of the CDRs of the invention into a human antibody scaffold or backbone. Humanized antibodies are well known in the art and any method of producing them that retains the CDRs of the invention may be employed.

The term “monoclonal antibody” or “mAb” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies and is not to be construed as produced by any specific preparation method. Monoclonal antibodies to be used in accordance with the methods provided herein, may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The mAb of the present invention may be of any immunoglobulin class including IgG, IgM, IgD, IgE or IgA. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multispecific antibodies formed from antibody fragments (e.g., including but not limited to, Db-Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three surfaces of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, delta, e, gamma, and micro, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies production is known in the art and is described in Natl. Acad. Sci. USA, 90:6444-6448 (1993).

The monoclonal antibodies of the invention may be prepared using methods well known in the art. Examples include various techniques, such as those in Kohler, G. and Milstein, C, Nature 256: 495-497 (1975); Kozbor et al, Immunology Today 4: 72 (1983); Cole et al, pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production, and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies, one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

In some embodiments, antibodies and portions thereof include but are not limited to: antibodies, fragments of antibodies, Fab and F(ab′)2, single-domain antigen-binding recombinant fragments and natural nanobodies. In some embodiments, the antigen binding fragment is selected from the group consisting of a Fv, Fab, F(ab′)2, scFv, scFv₂ or a scFv₄ fragment.

In some embodiments, the present invention provides nucleic acid sequences encoding the antibodies or antigen binding portions of the present invention.

For example, the polynucleotide may encode an entire immunoglobulin molecule chain, such as a light chain or a heavy chain. A complete heavy chain includes not only a heavy chain variable region (VH) but also a heavy chain constant region (CH), which typically will comprise three constant domains: CH1, CH2 and CH3; and a “hinge” region. In some situations, the presence of a constant region is desirable.

Other polypeptides which may be encoded by the polynucleotide include antigen-binding antibody fragments such as single domain antibodies (“dAbs”), Fv, scFv, Fab′ and CHI and CK or CL domain has been excised. As minibodies are smaller than conventional antibodies they should achieve better tissue penetration in clinical/diagnostic use but being bivalent they should retain higher binding affinity than monovalent antibody fragments, such as dAbs. Accordingly, unless the context dictates otherwise, the term “antibody” as used herein encompasses not only whole antibody molecules, but also antigen-binding antibody fragments of the type discussed above. Each framework region present in the encoded polypeptide may comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

Suitably, the polynucleotides described herein may be isolated and/or purified. In some embodiments, the polynucleotides are isolated polynucleotides.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring”.

In some embodiments, the antibody comprises an IgG4. In some embodiments, the antibody comprises an IgG2. In some embodiments, the antibody comprises an IgG2 or an IgG4. In some embodiments, the antibody comprises an IgG1, IgG2, IgG3 or IgG4. In some embodiments, the antibody does not comprise an IgG1. In some embodiments, the antibody does not comprise an IgG3. In some embodiments, the antibody does not comprise an IgG1 or IgG3. In some embodiments, the antibody comprises a mutated IgG1 and/or IgG3, wherein the mutation inhibits induction of ADCC, CDC or both. In some embodiments, the mutation is in the FcRgamma binding motif.

According to another aspect, there is provide a pharmaceutical composition comprising the agent of the invention.

In some embodiments, the pharmaceutical composition comprises a pharmaceutically acceptable carrier, excipient or adjuvant. In some embodiments, the pharmaceutical composition is formulated for systemic administration. In some embodiments, the pharmaceutical composition is formulated for intratumoral administration. In some embodiments, the pharmaceutical composition is formulated for administration to a subject. In some embodiments, the subject is a human.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

According to another aspect, there is provided a kit comprising an antibody of the invention or a pharmaceutical composition of the invention.

According to another aspect, there is provided a kit comprising an agent that bind to EPHB2 on a cell and inhibits a function of EPHB2 or a pharmaceutical composition comprising the agent and an immunotherapy.

In some embodiments, the kit further comprises an immunotherapy. In some embodiments, the immunotherapy is another immunotherapy. In some embodiments, the kit further comprises a pharmaceutical composition. In some embodiments, the pharmaceutical composition comprises an immunotherapy. In some embodiments, the agent is an antibody. In some embodiments, the kit further comprises a label stating the antibody of the invention or the pharmaceutical composition of the invention is for use with the immunotherapy. In some embodiments, the kit further comprises a label stating the antibody or the pharmaceutical composition comprising the antibody is for use with the immunotherapy. In some embodiments, the kit further comprises a label stating the immunotherapy is for use with the antibody or pharmaceutical composition of the invention. In some embodiments, the kit further comprises a label stating the immunotherapy is for use with the antibody or pharmaceutical composition comprising the antibody. In some embodiments, the kit is for use in a method of the invention. In some embodiments, the pharmaceutical composition is for use in a method of the invention.

According to another aspect, there is provided a method for producing an agent that enhances immunotherapy, the method comprising:
a. obtaining an agent that binds to an EPHB2 extracellular domain or a fragment thereof, assaying the efficacy of an immunotherapy on cancer cells treated with the agent, and selecting at least one agent that enhances the efficacy of the immunotherapy; or
b. culturing a host cell comprising one or more vectors comprising a nucleic acid sequence encoding an agent, wherein the nucleic acid sequence is that of an agent that was selected by:
   i. obtaining an agent that binds to an EPHB2 extracellular domain or a fragment thereof;
   ii. assaying the efficacy of an immunotherapy on cancer cells treated with the agent; and
   iii. selecting at least one agent that enhances the efficacy of the immunotherapy;
thereby producing an agent that enhances an immunotherapy.

In some embodiments, the method further comprises assaying EPHB2 function in the presence of the obtained agent. In some embodiments, the method comprises selecting an agent that inhibits EPHB2 function. In some embodiments, the method comprises selecting an agent that downregulates EPHB2 expression. In some embodiments, the method comprises selecting an agent that inhibits and/or blocks EPHB2 downstream signaling. In some embodiments, the method comprises selecting an agent that inhibits and/or blocks downstream signaling by an EPHB2 ligand. In some embodiments, the method comprises selecting an agent that blocks or inhibits binding of a ligand to EPHB2.

In some embodiments, assaying efficacy of an immunotherapy comprising measuring cancer cell killing. In some embodiments, assaying efficacy of an immunotherapy comprises measuring immune cell activation. In some embodiments, immune cell activation is measured by measuring expression of 41BB. In some embodiments, assaying immunotherapy comprises administering the immunotherapy to immune cells and contacting the immune cells with cancer cells treated with the agent. In some embodiments, the immune cells are PBMCs. In some embodiments, the immune cells are T cells. In some embodiments, the immune cells are TILs.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: siRNA Knockdown of EPHB2 with Anti-PD-1 Therapy

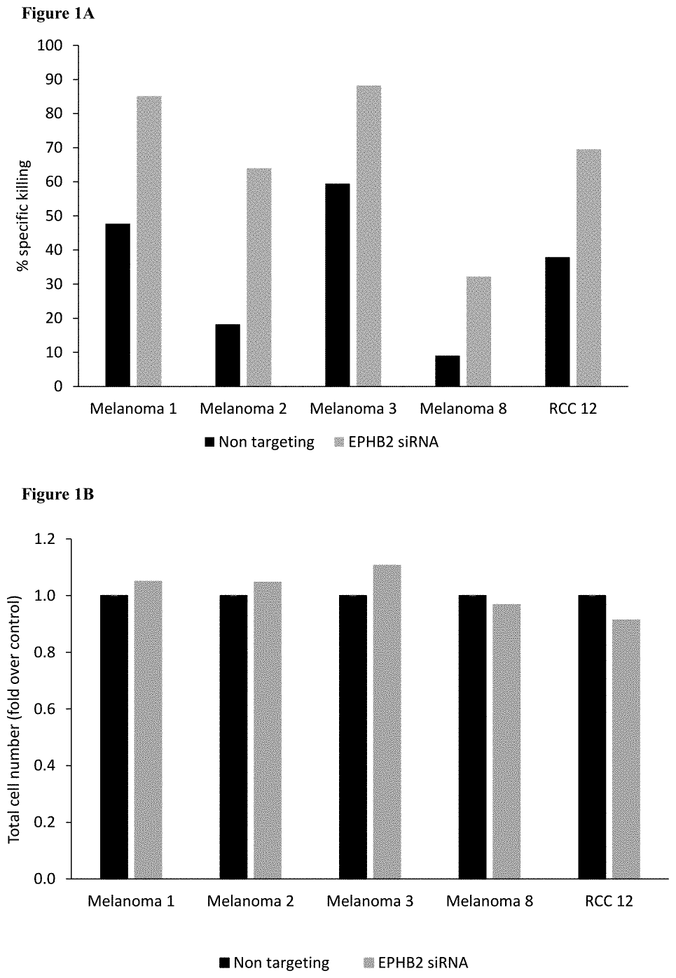

In order to test the inhibitory effect exerted by EPHB2, a pool of gene specific siRNAs (Dharmacon, L-003122-00) was transfected into melanoma or RCC cells using a reverse transfection method. A control, non-targeting, siRNA (Dharmacon, D-001810-10) was also transfected. After 48 hours for melanoma cells and 24 hours for RCC cells, IFNγ was added to the plates for overnight culturing. Autologous TILs were pre-incubated for one hour with 20 μg/ml of anti-PD-1 mAb (Nivolumab, BMS) and added to a 24 hour culture with the melanoma cells or a 48 hour culture with the RCC cells. Specific lysis of cancer cells was assessed by LDH release assay (Cat. G7891, Promega). FIG. 1A shows that knockdown of EPHB2 resulted in, on average, an 158% increase in specific killing of melanoma cells as compared to cells transfected with non-targeting siRNA. RCC cell killing was increased by 83%. Importantly, the anti-EPHB2 siRNA alone, without the addition of TILs, did not have a significant effect on total cell numbers (FIG. 1B).

### Example 2: Anti-EPHB2 Blocking Antibody in Combination with Anti-PD-1 Therapy

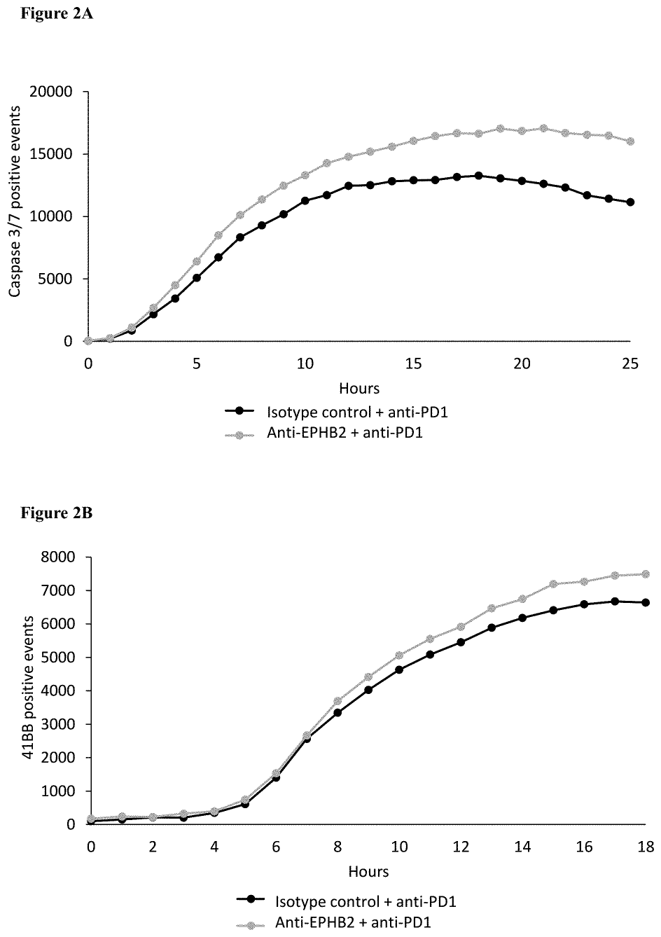

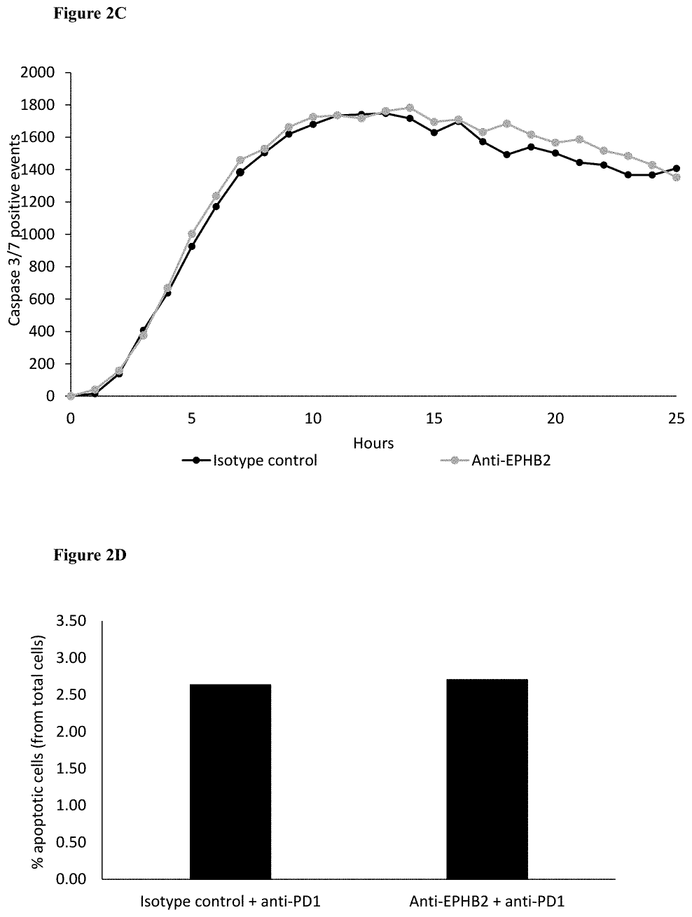

Next blocking of EPHB2 in conjunction with immune checkpoint blockade was investigated. Nuclear-RFP expressing melanoma cells were seeded for overnight incubation, in the presence of IFNγ, to allow them to attach. Melanoma cells were incubated for 1 hour at 37° C. with 20 μg/ml anti-EPHB2 antibody (BD Biosciences, clone 2H9) or 20 μg/ml isotype control antibody (BD Biosciences, clone MOPC-31C). Simultaneously, autologous TILs were incubated for 1 hour on ice with 20 μg/ml of anti-PD-1 mAb (Nivolumab, BMS). After the 1-hour pre-incubations with anti-PD-1, TILs were added to the cancerous cells. Two experiments were run on these mixed cells. In the first, CellEvent Caspase-3/7 Green Detection Reagent (Invitrogen, USA) was added to the culture. In the second, anti-41BB-APC antibody (Biolegend, USA) was added. Plates were placed in the Incucyte ZOOM system (ESSEN Bioscience, USA) and scanned every hour. Caspase 3/7 positive events, reflecting specific killing of cancerous cells, were counted, as was the expression of the activation marker 41BB on the TILs. FIGS. 2A-2B show that anti-EPHB2 in combination with anti-PD-1 treated TILs was superior to the anti-PD-1 treated TILs alone. Specifically, the addition of anti-EPHB2 resulted in a 44% increase in total melanoma cell killing after 25 hours (FIG. 2A), and a 13% increase in 41BB expression after 18 hours (FIG. 2B) as compared to cells treated with an isotype control. Importantly, in the absence of TILs, combined administration of anti-EPHB2 and anti-PD-1 had no effect on cell survival (FIG. 2D). Moreover, anti-EPHB2 alone had no effect on TIL cytotoxicity, suggesting that it exerts its effect only in combination with anti-PD-1 blockade of immune cells (FIG. 2C).

The combination therapy was found to be effective also in colon cancer. A newly harvested colon carcinoma was digested according to the following protocol. Tumor tissue was cut into small chunks and digested in media containing. gentamicin, DNase 3000 U/ml, and collagenase 0.1 g/ml. Tumor chunks were stirred at 37° C., in a container using a magnetic stirrer, until the cell dispersion is adequate. Cell suspension was then filtered through a sterile cell strainer, cells were pelleted by centrifugation, resuspended in PBS and pelleted again. Total viable nucleated cell number was determined, and cells were washed again and pelleted followed by re-suspension in growth medium.

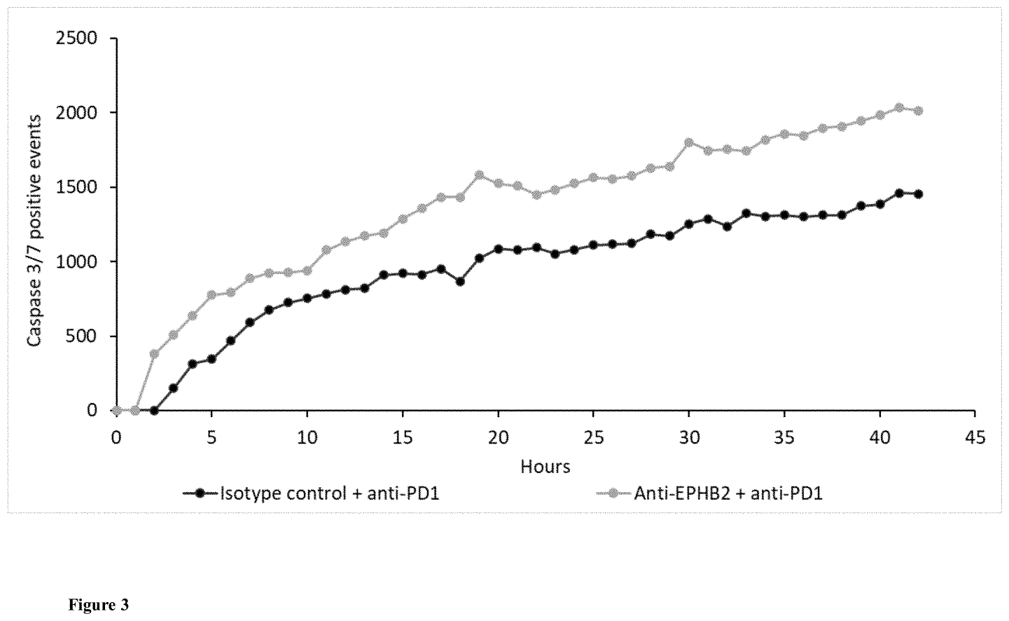

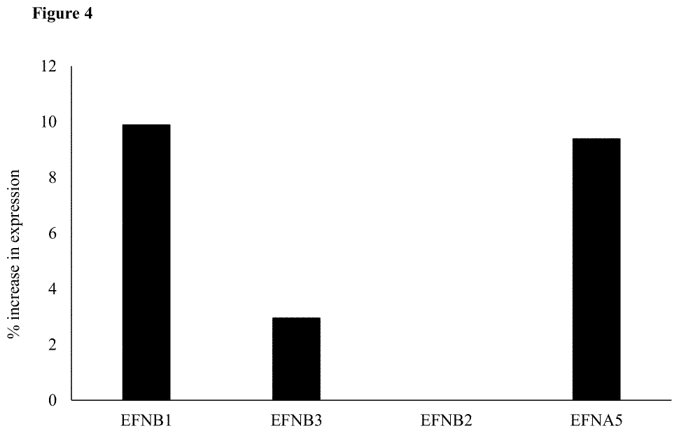

The colon carcinoma cells, consisting of the tumor cells as well as cells from the microenvironment of the tumor, were seeded in tissue culture plates in the presence of an anti-PD-1 antibody in combination with either the anti-EPHB2 antibody or an isotype control. Autologous PBMCs from the same patient as provided the carcinoma were immediately added to the cell culture. CellEvent Caspase-3/7 Green Detection Reagent was again used to assess cell killing. The plates were placed in an Incucyte Zoom system and scanned every 60 minutes. Killing was calculated by subtracting the positive events in PBMC only wells from the positive events in cancer+PBMC wells. As can be seen in FIG. 3, the addition of the anti-EPHB2 antibody potentiated the anti-PD-1 effect resulting in increased cancer cell killing (˜38% increase). As the anti-EPHB2 antibody alone (without addition of anti-PD-1 antibody) was shown not to effect cancer cell viability, the increased potency of the immunotherapy is due to a synergistic effect between EPHB2 blockade and PD-1 blockade.

### Example 3: Effect of Anti-PD-1 Therapy on Expression of EPH Ligands

It was hypothesized that the synergism between the anti-EPHB2 antibody and anti-PD-1 antibody could be due to an effect of PD-1 blockade on expression of EPH ligands. To test this, TILs were cultured with or without addition of anti-PD-1 antibody and the expression of four representative EPH ligands was measured. It was observed that anti-PD-1 antibody increased the expression of EFNB1 and EFNA5 and to a lesser extent EFNB3, while EFNB2 expression was unchanged (FIG. 4). Expression of EPHB2 by these cells was also unchanged (data not shown). Without being bound to any one particular mechanism, it may be that PD-1 blockade induces increased expression of EPH ligands by some immune cells which enhances the protective effect provided by EPHB2 on cancer cells. Thus, blockade of EPHB2 in combination with PD-1 blockade has an enhanced, synergistic effect.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of treating, preventing or ameliorating cancer in a subject in need thereof, the method comprising:
a. decreasing Ephrin type-B receptor 2 (EPHB2) function in a cell of said cancer; and
b. administering to said subject an immunotherapy,
thereby treating, preventing or ameliorating cancer in a subject.

2. (canceled)

3. (canceled)

4. The method of claim 1, wherein said cancer is any one of:
a. a solid tumor;
b. treatable by said immunotherapy;
c. expressing EPHB2;
d. selected from skin cancer, kidney cancer and colon cancer; and
e. selected from melanoma, renal cell carcinoma (RCC) and colon carcinoma.

5. (canceled)

6. (canceled)

7. (canceled)

8. (canceled)

9. The method of claim 1, wherein said subject is naïve to immunotherapy, has been previously treated by said immunotherapy, or has been treated by an immunotherapy other than said immunotherapy.

10. The method of claim 1, wherein said decreasing EPHB2 function comprises decreasing EPHB2 expression in said cell of said cancer or administering a pharmaceutical composition comprising a regulatory nucleic acid molecule that bind to an EPHB2 mRNA or the EPHB2 genomic locus.

11. (canceled)

12. The method of claim 1, wherein said decreasing function comprises administering a pharmaceutical composition comprising an agent that binds specifically to EPHB2.

13. The method of claim 12, wherein binding of said agent to EPHB2
a. induces reduced expression of EPHB2 on said cell surface;
b. induces degradation of said EPHB2;
c. blocks signaling through said EPHB2;
d. blocks binding of said EPHB2 to a ligand;
e. blocks binding of said EPHB2 to a B class ephrin, an A class ephrin or both;
f. blocks binding of said EPHB2 to any one of Ephrin B1 (EFNB1), Ephrin B3 (EFNB3) and Ephrin A5 (EFNA5);
g. blocks signaling through a ligand of EPHB2;
h. blocks signaling through a B class ephrin, an A class ephrin or both; or
i. blocks signaling through any one of EFNB1, EFNB3 and EFNA5.

14. (canceled)

15. (canceled)

16. The method of claim 12, wherein said agent is an EPHB2 blocking antibody.

17. The method of claim 12, wherein said agent is not coupled to a cytotoxic moiety.

18. The method of claim 1, wherein said immunotherapy comprises immune checkpoint blockade or adoptive T cell therapy.

19. The method of claim 18, wherein said immune checkpoint blockade comprises any one of:
a. administration of said immune checkpoint blockade to said subject;
b. treating ex vivo immune cells with said immune checkpoint blockade and administering said treated immune cells to said subject;
c. treating peripheral blood mononuclear cells (PBMCs), T cells, tumor infiltrating lymphocytes (TILs), natural killer cells (NK cells) or macrophages with said immune checkpoint blockade and administering said treated cells to said subject; and
d. blockade of an immune checkpoint protein selected from: PD-1, PD-L1, PD-L2, CD80, CD86, VISTA, CD275, CD276, VTCN1, HHLA2, CD96, CD155, TIGIT, CD112R, CD112, CD200, CD200R, CTLA4, LAG3, FGL1, TIM3, CEACAM-1, Gal-9, HMGB1, Butyrophilin family members, HVEM, or BTLA.

20. (canceled)

21. (canceled)

22. (canceled)

23. (canceled)

24. The method of claim 1, wherein said immunotherapy is anti-PD-1 antibody therapy.

25. The method of claim 1, wherein said decreasing EPHB2 function improves the efficacy of said immunotherapy.

26. The method of claim 1, wherein said treating, preventing or ameliorating cancer or said enhancing immunotherapy comprises increasing immune activation of an immune cell in said subject.

27. (canceled)

28. A kit comprising
a. an antibody that binds to EPHB2 on a cell and inhibits a function of said EPHB2 or a pharmaceutical composition comprising said antibody; and
b. a pharmaceutical composition comprising an immunotherapy.

29. The kit of claim 28,
a. further comprising a label stating said antibody or the pharmaceutical composition comprising said antibody is for use with said immunotherapy;
b. wherein said immunotherapy comprises immune checkpoint blockade;
c. wherein said immune checkpoint blockade comprises administering a pharmaceutical composition comprising an antibody that binds to and inhibits PD-1, PD-L1, PD-L2, CD80, CD86, VISTA, CD275, CD276, VTCN1, HHLA2, CD96, CD155, TIGIT, CD112R, CD112, CD200, CD200R, CTLA4, LAG3, FGL1, TIM3, CEACAM-1, Gal-9, HMGB1, Butyrophilin family members, HVEM, or BTLA; or
d. wherein said pharmaceutical composition comprises an antibody that binds to and inhibits PD-1.

30. (canceled)

31. (canceled)

32. (canceled)

33. A method for producing an agent that enhances an immunotherapy, the method comprising:
obtaining an agent that binds to an EPHB2 extracellular domain or a fragment thereof, assaying the efficacy of an immunotherapy on cancer cells treated with said agent, and selecting at least one agent that enhances said efficacy of said immunotherapy; or
culturing a host cell comprising one or more vectors comprising a nucleic acid sequence encoding an agent, wherein the nucleic acid sequence is that of an agent that was selected by:
i. obtaining an agent that binds to an EPHB2 extracellular domain or a fragment thereof;
ii. assaying the efficacy of an immunotherapy on cancer cells treated with said agent; and
iii. selecting at least one agent that enhances said efficacy of said immunotherapy;
thereby producing an agent that enhances an immunotherapy.

34. The method of claim 33, further comprising assaying EPHB2 function in the presence of said obtained agent and selecting an agent that inhibits EPHB2 function.

35. The method of claim 34, wherein said EPHB2 function is selected from:
a. EPHB2 downstream signaling;
b. EPHB2 ligand binding;
c. downstream signaling of an EPHB2 ligand;
d. downstream signaling of a B class ephrin or an A class ephrin; and
e. downstream signaling of any one of Ephrin B1 (EFNB1), Ephrin B3 (EFNB3) and Ephrin A5 (EFNA5).

36. (canceled)

37. (canceled)

38. The method of claim 33, wherein said immunotherapy comprises immune checkpoint blockade or inhibition of an immune checkpoint protein selected from: PD-1, PD-L1, PD-L2, CD80, CD86, VISTA, CD275, CD276, VTCN1, HHLA2, CD96, CD155, TIGIT, CD112R, CD112, CD200, CD200R, CTLA4, LAG3, FGL1, TIM3, CEACAM-1, Gal-9, HMGB1, Butyrophilin family members, HVEM, and BTLA.

39. (canceled)

40. (canceled)

41. The method of claim 1, consisting essentially of decreasing EPHB2 function in said cell of said cancer, and administering said immunotherapy.
